(19) United States
(12) Patent Application Publication
(10) Pub. No.: US 2009/0099370 A1
(43) Pub. Date: Apr. 16, 2009

(54) Crystalline Form of Perindopril Erbumine

(75) Inventors: Ulrich Griesser, Axams (AT); Verena Niederwanger, Bruneck (IT)

Correspondence Address: SANDOZ INC, 506 CARNEFIE CENTER, PRINCETON, NJ, 08540, US

(73) Assignee: Sandoz AG, Basel (CH)

(21) Appl. No.: 11/990,351

(22) PCT Filed: Aug. 10, 2006
(86) PCT No.: PCT/EP2006/007923

§ 371 Date: Feb. 12, 2008

(30) Foreign Application Priority Data
Aug. 12, 2005 (SI) P200500231

(51) Int. Cl. C07D 209/42

(52) U.S. Cl. 548/452

(57) ABSTRACT

The present invention relates to new crystalline form of the ACE inhibitor perindopril and processes for the preparation thereof.

The present invention relates to new crystalline form of perindopril erbumine.

Perindopril and its pharmaceutically acceptable salts are known as angiotensin converting enzyme inhibitors and are used in the treatment of cardiovascular diseases, especially in the treatment of hypertension and heart failure. Perindopril is chemically known as (2S,3aS,7aS)-((2-(1-(ethoxycarbonyl)-(S)-butylamino)-(S)-propionyl)octahydro-indole-2-carboxylic acid and can be represented by formula (I).

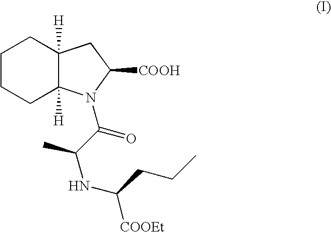

Perindopril was first disclosed in EP 0049658 B1 and U.S. Pat. No. 4,508,729 as optically pure S,S,S,S,S isomer and in the form of sodium salt. Numerous later patents and patent applications, such as EP 0308341 B1, EP 1279665 A1, EP 1333026 A1, WO 2004/099236 describe various processes for the preparation of perindopril.

Perindopril in a non-salted form is an oily, amorphous or hardly crystalline material depending on a process for the preparation and presence of impurities. In addition to the by-products formed during the process of the preparation thereof, also degradation products of perindopril, such as diketopiperazines and perindoprilate, are present as impurities in crude perindopril.

Pharmaceutically acceptable compounds have to be obtained in high purity and have to be stable for long periods. Additionally, it is important that such compounds have valuable characteristics of isolation, such as filtration and drying, to be produced at industrial scale.

Hitherto only tert-butylamine salt of perindopril, i.e. perindopril erbumine, firstly disclosed in EP 0308341, has enough good crystalline properties to be used for pharmaceutical purposes (i.e. having well defined and stable physical properties). Perindopril erbumine may be obtained in different crystalline forms depending on crystallization conditions, e.g. solvent system, perindopril erbumine concentration and cooling kinetic. EP 1296947 B1 discloses crystallization of perindopril erbumine crystalline form α from ethyl acetate, EP 1294689 A discloses crystallization of perindopril erbumine crystalline form β from dichloromethane or ethyl acetate, EP 1296948 B1 discloses crystallization of perindopril erbumine crystalline form γ from chloroform and WO 2004/113293 discloses crystallization of perindopril erbumine crystalline form δ and crystalline form ε. Crystalline form ε is obtained by crystallization from tert-butyl methyl ether containing 1.5 to 2.5% (vol/vol) of water. whereas crystalline form δ is obtained from form ε by azeotropic distillation.

However, there is a continuing need to obtain new crystalline form of perindopril erbumine having improved characteristics in term of isolation and purification.

A first object of the present invention is related to new crystalline form of perindopril erbumine, named form D, having a powder x-ray diffraction pattern comprising the following characteristic reflection angles 2θ: 5.3±0.2°, 10.7±0.2°, 16.1±0.2°, 24.4±0.2° and 27.0±0.2°.

New crystalline form D of perindopril erbumine has a powder x-ray diffraction pattern as depicted in FIG. 1 and shows the following characteristic 2θ angles:

| Angle 2θ | d value | Intensity |
|---|---|---|
| (°) | (Angstrom) | (%) |
| 5.3 | 16.61 | 7 |
| 8.4 | 10.52 | 8 |
| 9.4 | 9.37 | 45 |
| 10.7 | 8.29 | 6 |
| 14.7 | 6.01 | 11 |
| 15.5 | 5.71 | 23 |
| 16.1 | 5.52 | 100 |
| 16.8 | 5.29 | 7 |
| 17.7 | 5.00 | 10 |
| 18.4 | 4.83 | 8 |
| 19.4 | 4.58 | 6 |
| 19.7 | 4.51 | 5 |
| 20.1 | 4.41 | 5 |
| 21.2 | 4.20 | 13 |
| 21.5 | 4.14 | 48 |
| 21.7 | 4.09 | 18 |
| 23.0 | 3.86 | 5 |
| 23.5 | 3.78 | 8 |
| 24.4 | 3.64 | 11 |
| 25.8 | 3.45 | 5 |
| 27.0 | 3.30 | 13 |
| 27.4 | 3.25 | 6 |
| 28.7 | 3.11 | 4 |
| 29.0 | 3.08 | 3 |
| 30.4 | 2.94 | 3 |
| 32.3 | 2.77 | 6 |
| 34.1 | 2.62 | 2 |
| 35.3 | 2.54 | 4 |
| 36.8 | 2.44 | 2 |

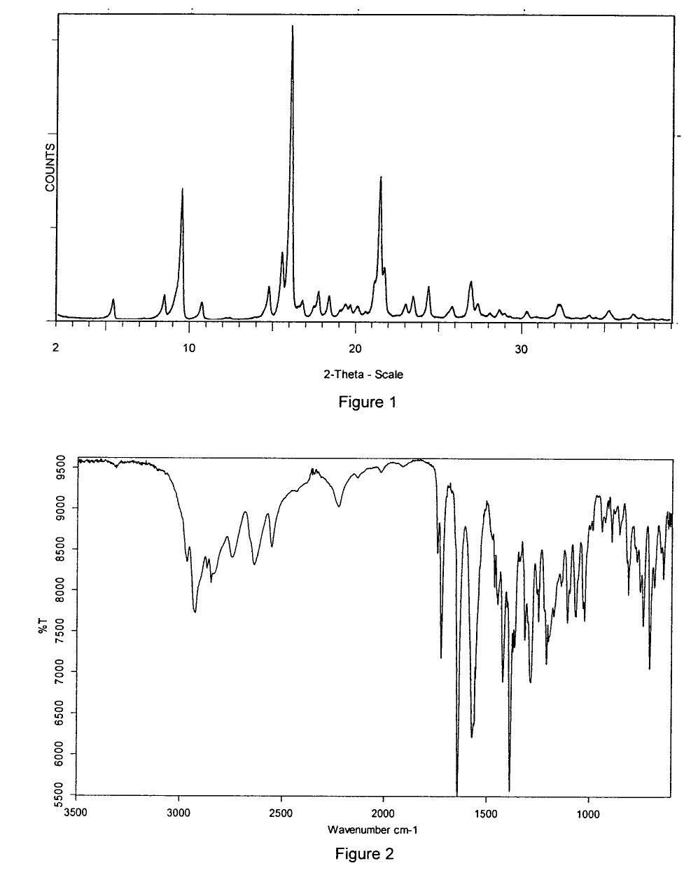

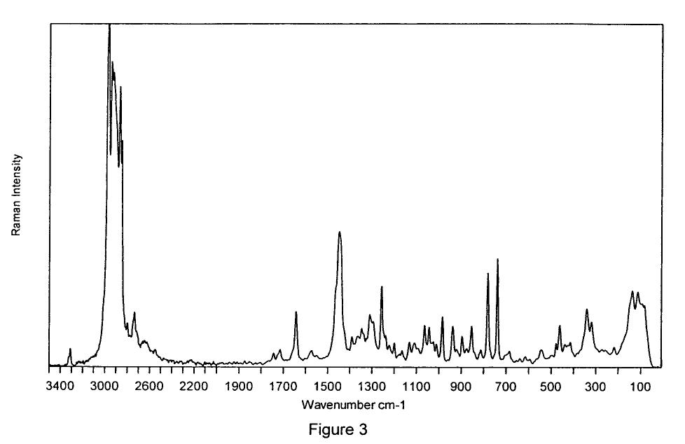

New crystalline form D of perindopril erbumine may be further characterized by IR spectrum as depicted in FIG. 2 having bands at 2926 cm⁻¹, 2747 cm⁻¹, 2640 cm⁻¹, 2555 cm⁻¹, 1745 cm⁻¹, 1725 cm⁻¹, 1642 cm⁻¹, 1572 cm⁻¹, 1557 cm⁻¹, 1423 cm⁻¹, 1388 cm⁻¹, 1374 cm⁻¹, 1287 cm⁻¹, 1248 cm⁻¹, 1210 cm⁻¹, 1139 cm⁻¹, 1107 cm⁻¹, 1065 cm⁻¹, 1025 cm⁻¹, 986 cm⁻¹, 739 cm⁻¹, 707 cm⁻¹.

New crystalline form D of perindopril erbumine may be further characterized by the following characteristic peaks in Raman spectrum: 2969 cm⁻¹, 2924 cm⁻¹, 1573 cm⁻¹, 1450 cm⁻¹ and 542 cm⁻¹.

New crystalline form D of perindopril erbumine may be further characterized by Raman spectrum as depicted in FIG. 3 with characteristic peaks at: 3310 cm⁻¹, 2969 cm⁻¹, 2941 cm⁻¹, 2924 cm⁻¹, 2869 cm⁻¹, 2851 cm⁻¹, 2798 cm⁻¹, 2735 cm⁻¹, 1643 cm⁻¹, 1573 cm⁻¹, 1450 cm⁻¹, 782 cm⁻¹, 739 cm⁻¹, 542 cm⁻¹, 138 cm⁻¹, 115 cm⁻¹.

The Applicant has surprisingly found that new crystalline form D of perindopril erbumine has valuable characteristic in term of isolation and purification.

The new crystalline form D of perindopril erbumine according to the invention is obtained in a higher purity compared to the crystalline form α or any other known crystalline form of perindopril erbumine, when both forms are prepared from the same crude perindopril and using the same number of recrystallization steps.

Another object of the present invention is related to processes for the preparation of new crystalline form of perindopril erbumine. New crystalline form D of perindopril erbumine may be prepared from any other crystalline form of perindopril erbumine or from solution of perindopril erbumine.

In one embodiment, the present invention relates to a process (a) for the preparation of new crystalline form D of perindopril erbumine comprising the steps of:
(a1) suspending perindopril erbumine in dichloromethane,
(a2) heating the suspension up to 40° C.,
(a3) cooling down obtained solution slowly to 20-25° C., preferably at a rate of about 10° C./h and,
(a4) isolating new crystalline form D, preferably by filtration under reduced pressure, preferably at about 10 mbar, followed by drying.

In another embodiment, the present invention relates to a process (b) for the preparation of new crystalline form D of perindopril erbumine comprising the steps of:
(b1) suspending perindopril erbumine in dichloromethane,
(b2) heating the suspension up to 40° C.,
(b3) cooling down obtained solution in an ice-bath and,
(b4) isolating new crystalline form D, preferably by filtration under reduced pressure, preferably at about 10 mbar, followed by drying.

In another embodiment, the present invention relates to a process (c) for the preparation of new crystalline form D of perindopril erbumine comprising the steps of:
(c1) suspending perindopril erbumine in acetonitrile,
(c2) heating the suspension up to 70° C.,
(c3) cooling down obtained solution slowly to 20-25° C., preferably at a rate of about 10° C./h and,
(c4) isolating new crystalline form D, preferably by filtration under reduced pressure, preferably at about 10 mbar, followed by drying.

In another embodiment, the present invention relates to a process (d) for the preparation of new crystalline form D of perindopril erbumine comprising a lyophilization of about 5% perindopril erbumine solution in water. For example, said solution is freezed with liquid nitrogen and freeze drying is performed at −20° C. and at a pressure of about 0.2 mbar over three days.

In another embodiment, the present invention relates to a process (e) for the preparation of new crystalline form D of perindopril erbumine comprising the steps of:
(e1) moistening perindopril erbumine with water and,
(e2) drying obtained paste at temperature from 20-25° C. and a relative humidity below 40%.

In another embodiment, the present invention relates to a process (f) for the preparation of new crystalline form D of perindopril erbumine comprising the steps of:

(f1) suspending perindopril erbumine in t-butylmethyl ether,
(f2) adding sufficient amount of water to induce dissolution, e.g. about 1-5% (vol/vol) of water, preferably about 3% (vol/vol) of water is added,
(f3) heating the suspension up to 40° C.,
(f4) cooling down obtained solution slowly to 20-25° C., preferably at a rate of about 10° C./h and,
(f5) isolating new crystalline form D, preferably by filtration under reduced pressure, preferably at about 10 mbar, followed by drying.

In another embodiment, the present invention relates to a process (g) for the preparation of new crystalline form D of perindopril erbumine comprising the steps of:
(g1) suspending perindopril erbumine in t-butylmethyl ether,
(g2) adding sufficient amount of water to induce dissolution, e.g. about 1-5% (vol/vol) of water, preferably about 3% (vol/vol) of water is added,
(g3) heating the suspension up to 40° C.,
(g4) cooling down obtained solution quickly to about 5° C. and,
(g5) isolating new crystalline form D, preferably by filtration under reduced pressure, preferably at about 10 mbar, followed by drying.

Another object of the present invention is related to any of the processes as described above, wherein in a further step the new crystalline form D of perindopril erbumine as obtained according to one of the processes as described previously is formulated into a pharmaceutically acceptable dosage form, in particular wherein said dosage form is a tablet, pill, capsule or injectable.

Another object of the present invention is related to use of new perindopril erbumine crystalline form D in the process for the preparation of pure crystalline form α or any other known crystalline form of perindopril erbumine.

Another object of the present invention is related to a process for the preparation of pure crystalline form α or any other known crystalline form of perindopril erbumine comprising a step of preparing new crystalline form D of perindopril erbumine.

“Pure” crystalline form of perindopril erbumine in the present invention means crystalline form of perindopril erbumine having more than 95%, preferably more than 99%, more preferably more than 99.5%, most preferably more than 99.8%, of chromatographic purity.

Another object of the present invention is related to pharmaceutical compositions comprising a therapeutically effective amount of new crystalline form D of perindopril erbumine with one or more pharmaceutically acceptable carriers or other excipients.

A therapeutically effective amount of perindopril salt is the amount of perindopril salt which comprises an amount of perindopril which is appropriate in a dosage form useful to treat hypertension or cardiovascular diseases. In general, a pharmaceutically effective amount of perindopril is 1 to 15 mg of perindopril, preferably 2 to 8 mg.

Pharmaceutically acceptable excipients may be selected from the group consisting of binders, diluents, disintegrating agents, stabilizing agents, preservatives, lubricants, fragrances, flavoring agents, sweeteners and other excipients known in the field of the pharmaceutical technology. Preferably, carriers and excipients may be selected from the group consisting of hydroxypropylcellulose, lactose, microcrystalline cellulose, calcium carbonate, starch, colloidal silicone dioxide, sodium starch glycolate, talc, magnesium stearate, polyvinylpyrrolidone, and other excipients known in the field of the pharmaceutical technology.

Optionally, the pharmaceutical compositions of the invention may be combination products comprising one or more additional pharmaceutically active components in addition to perindopril. Preferably, an additional pharmaceutically active component is a diuretic, e.g. indapamide.

Suitable pharmaceutical compositions are solid dosage forms, such as tablets with immediate release or sustained release of the active principle, effervescent tablets or dispersion tablets and capsules.

The pharmaceutical compositions may be prepared by methods known in the field of the pharmaceutical technology.

Another object of the present invention is related to the use of new crystalline form D of perindopril erbumine for the preparation of a pharmaceutical composition for use in the treatment of cardiovascular diseases, e.g. hypertension or heart failure.

Another object of the present invention is related to a method for the treatment of cardiovascular diseases, e.g. hypertension or heart failure, comprising administering a therapeutically effective amount of new crystalline form D of perindopril erbumine.

The following examples illustrate the invention, but do not limit it in any way.

FIG. 1 depicts a powder x-ray diffraction pattern of new crystalline form D of perindopril.

FIG. 2 depicts IR spectrum of new crystalline form D of perindopril.

FIG. 3 depicts Raman spectrum of new crystalline form D of perindopril.

### EXAMPLE 1

### Preparation of Crystalline Form D of Perindopril Erbumine

0.25 g of perindopril erbumine form α are suspended in 5 ml of dichloromethane and the suspension is heated up to 40° C. The clear solution is cooled down to room-temperature at a rate of about 10K/h. The yield is filtered under reduced pressure (10 mbar) and air-dried. Yield: 0.23 g (92%)

### EXAMPLE 2

### Preparation of Crystalline Form D of Perindopril Erbumine

0.25 g perindopril erbumine form α are suspended in 5 ml of dichloromethane and the suspension is heated up to 40° C. The clear solution is cooled in an ice-bath. The colorless crystals are filtered under reduced pressure (about 10 mbar) and air-dried. Yield: 0.18 g (72%)

### EXAMPLE 3

### Preparation of Crystalline Form D of Perindopril Erbumine

0.25 g perindopril erbumine form α are suspended in 12 ml of acetonitrile and the suspension is heated up to 70° C. The clear solution is cooled down to room-temperature at a rate of about 10K/h. The colorless crystals are filtered under reduced pressure (about 10 mbar) and air-dried. Yield: 0.16 g (64%)

### EXAMPLE 4

### Preparation of Crystalline Form D of Perindopril Erbumine by Lyophilization of Perindopril Erbumine Solution

5% perindopril erbumine solution in water is freezed with liquid nitrogen. Freeze drying is performed at −20° C. and at a pressure of about 0.2 mbar over three days.

### EXAMPLE 5

### Preparation of Crystalline Form D of Perindopril Erbumine

The dry perindopril erbumine is placed in a mortar and a few drops of water are added. The mixture is homogenized with a pistil and the resulting paste is dried at room temperature (20-25° C.) at a relative humidity below 40%.

### EXAMPLE 6

### Preparation of Crystalline Form D of Perindopril Erbumine

0.5 g perindopril erbumine form α are suspended in 5 ml of t-butylmethyl ether and three drops of water are added. The suspension is heated up to 40° C. and the clear solution is cooled down to room-temperature at a rate of about 10K/h. The clear crystals are filtered under reduced pressure (about 10 mbar) and air-dried. Yield: 0.44 g (88%).

### EXAMPLE 7

### Preparation of Crystalline Form D of Perindopril Erbumine

0.5 g perindopril erbumine form α are suspended in 5 ml of t-butylmethyl ether and three drops of water are added. The suspension is heated up to 40° C. and the clear solution is quickly cooled to about 5° C. The clear crystals are filtered under reduced pressure (about 10 mbar) and air-dried. Yield: 0.46 g (92%).

Analytical data in examples are achieved by the following hardware:

### Infrared Spectroscopy:

Fourier transform infrared (FTIR) spectra are recorded with a Brucker IFS 25 spectrometer (Brucker Analytische Messtechnik GmbH, Karlsruhe, D). Spectra over a range of 4000 to 400 cm⁻¹ with a resolution of 2 cm⁻¹ (64 scans) are recorded on KBr tablets (approximately 1.5 mg BD 104 per 275 mg KBr).

Most of the spectra are recorded with a Durasampler ATR on a Spectrum GX FT-IR-System spectrometer (Perkin Elmer, Norwalk Conn., USA). The spectra are recorded over a range of 4000 to 600 cm⁻¹ with a resolution of 2 cm⁻¹ (24 scans). The analysis is done with Spectrum v 2.00 software.

### Raman-Spectroscopy:

Raman spectra of the forms are recorded with a Brucker RFS 100 Raman spectrometer (Bruker Analytische Messtechnik GmbH, Karlsruhe, D), equipped with a Nd:YAG Laser (1064 nm) as excitation source and a liquid-nitrogen-cooled, high-sensitivity Ge-detector. The spectra from 3500 to 10 cm⁻¹ are recorded in aluminium sample holders at a laser power of 300 mW (64 scans per spectrum).

### Powder X-Ray Diffraction (PXRD):

The X-ray diffraction patterns are obtained using a Siemens D-5000 diffractometer (Bruker AXS, Karlsruhe, D) equipped with a theta/theta goniometer, a CuK_{α} radiation source, a Goebel mirror (Bruker AXS, Karlsruhe, D), a 0.15° soller slit collimator and a scintillation counter. The patterns are recorded at a tube voltage of 40 kV and a tube current of 35 mA, applying a scan rate of 0.005° 2θs⁻¹ in the angular range of 2 to 40° 2θ.

1. Crystalline form D of perindopril erbumine characterized by a powder x-ray diffraction pattern comprising the following characteristic diffraction angles (2θ): 5.3±0.2°, 10.7±0.2°, 16.1±0.2°, 24.4±0.2° and 27.0±0.2°.

2. Crystalline form D of perindopril erbumine according to claim 1 further characterized by a powder x-ray diffraction pattern comprising the following characteristic diffraction angles (2θ):

| Angle 2θ | d value | Intensity |
|---|---|---|
| (°) | (Angstrom) | (%) |
| 5.3 | 16.61 | 7 |
| 8.4 | 10.52 | 8 |
| 9.4 | 9.37 | 45 |
| 10.7 | 8.29 | 6 |
| 14.7 | 6.01 | 11 |
| 15.5 | 5.71 | 23 |
| 16.1 | 5.52 | 100 |
| 16.8 | 5.29 | 7 |
| 17.7 | 5.00 | 10 |
| 18.4 | 4.83 | 8 |
| 19.4 | 4.58 | 6 |
| 19.7 | 4.51 | 5 |
| 20.1 | 4.41 | 5 |
| 21.2 | 4.20 | 13 |
| 21.5 | 4.14 | 48 |
| 21.7 | 4.09 | 18 |
| 23.0 | 3.86 | 5 |
| 23.5 | 3.78 | 8 |
| 24.4 | 3.64 | 11 |
| 25.8 | 3.45 | 5 |
| 27.0 | 3.30 | 13 |
| 27.4 | 3.25 | 6 |
| 28.7 | 3.11 | 4 |
| 29.0 | 3.08 | 3 |
| 30.4 | 2.94 | 3 |
| 32.3 | 2.77 | 6 |
| 34.1 | 2.62 | 2 |
| 35.3 | 2.54 | 4 |
| 36.8 | 2.44 | 2 |

3. Crystalline form D of perindopril erbumine according to claim 1 further characterized by a powder x-ray diffraction pattern as depicted in FIG. 1.

4. Crystalline form D of perindopril erbumine according to claim 1 further characterized by IR spectrum as depicted in FIG. 2.

5. Crystalline form D of perindopril erbumine according to claim 1 further characterized by Raman spectrum having characteristic peaks at: 2969 cm⁻¹, 2924 cm⁻¹, 1573 cm⁻¹, 1450 cm⁻¹ and 542 cm⁻¹.

6. A process for the preparation of crystalline form D of perindopril erbumine according to claim 1 comprising steps of:
(a1) suspending perindopril erbumine in dichloromethane,
(a2) heating the suspension up to 40° C.,
(a3) cooling down obtained solution slowly to 20-25° C. and,
(a4) isolating said crystalline form.

7. A process for the preparation of crystalline form D of perindopril erbumine according to claim 1 comprising steps of:
(b1) suspending perindopril erbumine in dichloromethane,
(b2) heating the suspension up to 40° C.,
(b3) cooling down obtained solution in an ice-bath and,
(b4) isolating said crystalline form.

8. A process for the preparation of crystalline form D of perindopril erbumine according to claim 1 comprising steps of:
(c1) suspending perindopril erbumine in acetonitrile,
(c2) heating the suspension up to 70° C.,
(c3) cooling down obtained solution slowly to 20-25° C. and,
(c4) isolating said crystalline form.

9. A process for the preparation of crystalline form D of perindopril erbumine according to claim 1 comprising a lyophilization of 5% perindopril erbumine solution in water.

10. A process for the preparation of crystalline form D of perindopril erbumine according to claim 1 comprising steps of:
(e1) moistening perindopril erbumine with water and,
(e2) drying obtained paste at temperature from 20-25° C. and a relative humidity below 40%.

11. A process for the preparation of crystalline form D of perindopril erbumine according to claim 1 comprising steps of:
(f1) suspending perindopril erbumine in t-butylmethyl ether,
(f2) adding sufficient amount of water to induce dissolution,
(f3) heating the suspension up to 40° C.
(f4) cooling down obtained solution slowly to 20-25° C. and,
(f5) isolating said crystalline form.

12. A process for the preparation of crystalline form D of perindopril erbumine according to claim 1 comprising steps of:
(g1) suspending perindopril erbumine in t-butylmethyl ether,
(g2) adding sufficient amount of water to induce dissolution,
(g3) heating the suspension up to 40° C.
(g4) cooling down obtained solution quickly to about 5° C. and,
(g5) isolating said crystalline form.

13. Use of crystalline form D of perindopril erbumine according to claim 1 for the preparation of pure crystalline form α or any other known crystalline form of perindopril erbumine.

14. A process for the preparation of pure crystalline form α or any other known crystalline form of perindopril erbumine comprising a step of preparing crystalline from D of perindopril erbumine according to claim 1.

15. A process according to claim 6, wherein in a further step the obtained crystalline form D of perindopril erbumine is formulated into a pharmaceutically acceptable dosage form, in particular wherein said dosage form is a tablet, pill, capsule or injectable.

16. A pharmaceutical composition comprising crystalline form D of perindopril erbumine according to claim 1.

17. Use of new crystalline form D of perindopril erbumine according to claim 1 for the preparation of a pharmaceutical composition for use in the treatment of cardiovascular diseases.
